SEQUENCE LISTING (7 sequences)

SEQ ID NO: 1 - DNA, 368 bp, unidentified
  source 1..368: mol_type = unassigned DNA
cggtaggtgaacctgccgttggatcagttctagtttaataactaataccaacttctttttattaatcaaaaactaatacaattataaataactaacttcatcaaaaatcaagtctctaagcaatggatgtcttggctcctcacacgatgaagaacgtggcataatgtgttaagtaaccggagttgcatacatcatgacaggttaatctttgaatgcaaattgcgctaaacttggcttcggctgagaagcatgcgtgtaacagtacaacataatttataataattcttattctaagcgaataagtaaataaattataagacaaacttacgttgtctgctatacgcaggaagacccgctgaactgaagca

SEQ ID NO: 2 - DNA, 42 bp, synthetic construct
  source 1..42: mol_type = other DNA
gtcatgatgtatgcaactccggtcctcacacgatgaagaacg

SEQ ID NO: 3 - DNA, 48 bp, synthetic construct
  source 1..48: mol_type = other DNA
ggttaatctttgaatgcaaattgcgtgtactgttacacgcatgcttct

SEQ ID NO: 4 - DNA, 23 bp, synthetic construct
  source 1..23: mol_type = other DNA
acattatgccacgttcttcatcg

SEQ ID NO: 5 - DNA, 20 bp, synthetic construct
  source 1..20: mol_type = other DNA
tgcgctaaacttggcttcgg

SEQ ID NO: 6 - DNA, 18 bp, synthetic construct
  source 1..18: mol_type = other DNA
agcaatggatgtcttggc

SEQ ID NO: 7 - DNA, 19 bp, synthetic construct
  source 1..19: mol_type = other DNA
gcagacaacgtaagtttgt

(19) United States
(12) Patent Application Publication
(10) Pub. No.: US 2023/0340619 A1
(43) Pub. Date: Oct. 26, 2023

(54) METHODS OF DETECTING TRICHOMONAS TENAX

(71) Applicant: Adtalem Global Education Inc., Chicago, IL (US)

(72) Inventor: Chaoqun YAO, Urbandale, IA (US)

(21) Appl. No.: 18/106,394

(22) Filed: Feb. 6, 2023

Related U.S. Application Data
(60) Provisional application No. 63/307,513, filed on Feb. 7, 2022.

(51) Int. Cl. C12Q 1/6893

(52) U.S. Cl. CPC C12Q 1/6893; C12Q 2600/16

(57) ABSTRACT

Provided are methods for detecting T. tenax via LAMP assay. This newly developed LAMP assay has high sensitivity and specificity and provides a quick, cheap, easy and reliable method used for the detection of T. tenax that can be used as a point-of-care test in human and veterinary medicine as well as in epidemiological studies of T. tenax infections in humans, dogs and other mammals.

### RELATED PATENT APPLICATIONS

This patent application claims priority to U.S. Provisional Patent Application No. 63/307,513, filed on Feb. 7, 2022, entitled METHODS OF DETECTING TRICHOMONAS TENAX, naming Chaoqun YAO as inventor, and designated by Attorney Docket No. ADTAL-1002PROV. This application also is related to a PCT Application filed on Feb. 6, 2023, entitled METHODS OF DETECTING TRICHOMONAS TENAX, naming Chaoqun YAO as inventor, and designated by Attorney Docket No. ADTAL-1002PCT. The entire contents of the foregoing patent applications are incorporated herein by reference for all purposes.

### SEQUENCE LISTING

The instant application contains a Sequence Listing which has been submitted electronically in XML format and is hereby incorporated by reference in its entirety. Said XML copy, created on Mar. 22, 2023, is named ADTAL-1002_SL.xml and is 7,574 bytes in size.

### FIELD

The technology relates in part to methods and compositions for detecting Trichomonas tenax.

### BACKGROUND

Trichomonas tenax (T. tenax) is a flagellated protozoan that inhabits the human and canine oral cavity in patients with poor oral hygiene and periodontal disease. In addition, T. tenax, like a related human parasite, T. vaginalis, has been detected in the urogenital tract. However, despite the association of T. tenax with periodontal diseases and sexually transmitted diseases, the availability of fast and reliable methods for the detection of T. tenax is lacking. Thus, there is a need for such methods.

### SUMMARY

The present disclosure is directed in part to a method for detecting T. tenax comprising preparing a biological sample for detection; performing LAMP on the biological sample; and determining whether the biological sample comprises T. tenax.

In some embodiments, the biological sample comprises an oral swab or saliva or urine.

In some embodiments, the biological sample is diluted in PBS.

In any of the foregoing embodiments, neither DNA extraction nor cell boiling has been performed on the biological sample before the step of performing LAMP; in embodiments,

DNA extraction, boiling, cell boiling or any combination thereof has been performed on the biological sample before the step of performing LAMP.

In any of the foregoing embodiments, performing LAMP comprises at least one of optimized MgSO₄ concentration, temperature, and reaction time. In some embodiments, the optimized MgSO₄ concentration comprises 6 mM. In some embodiments, the optimized temperature comprises 60° C. In some embodiments, the optimized reaction time comprises 60 minutes.

In any of the foregoing embodiments, the determining step is at least 100-times more sensitive than conventional PCR.

In any of the foregoing embodiments, the determining step does not detect any one or more of the microbes comprising T. vaginalis, S. pyogenes, S. aureus, E. coli, E. faecalis, and C. albicans.

In any of the foregoing embodiments, the step of performing LAMP comprises use of the set of primers listed in TABLE 1 (SEQ ID NOS:2-7).

Provided herein is a method for detecting T. tenax comprising, preparing a biological sample for detection; performing LAMP on the biological sample; and determining whether the biological sample comprises T. tenax. Also provided herein is a method for detecting T. tenax comprising, preparing a biological sample for detection; analyzing the biological sample for the presence or absence of nucleic acid, such as DNA or RNA, from T. tenax; and determining whether the biological sample comprises T. tenax. The biological sample can be obtained from a suitable subject, e.g., a eukaryotic vertebrate. In some embodiments, the subject is a human. In embodiments, the subject is a canine.

Biological samples that can be analyzed according to any embodiments of the methods provided herein include, but are not limited to, blood, plasma, serum, urine, saliva, seminal fluid, lavages (e.g., bronchoalveolar, gastric, peritoneal, ductal, ear, arthroscopic), cervical fluid, cervicovaginal fluid, cerebrospinal fluid, vaginal fluid, breast fluid, breast milk, synovial fluid, semen, seminal fluid, sputum, cerebral spinal fluid, tears, mucus, interstitial fluid, follicular fluid, amniotic fluid, aqueous humor, vitreous humor, peritoneal fluid, ascites, sweat, lymphatic fluid, lung sputum or fractions or components thereof. In certain embodiments, the biological sample comprises an oral swab or saliva or urine.

In any embodiments of the methods provided herein, the biological sample can be diluted in PBS. In embodiments, neither DNA extraction nor cell boiling has been performed on the biological sample before the step of analyzing the DNA, such as by performing LAMP. In any embodiments of the methods provided herein, performing LAMP comprises at least one of optimized MgSO₄ concentration, optimized temperature, and optimized reaction time. In embodiments, the optimized MgSO₄ concentration is about or equal to 6 mM. In certain embodiments, the optimized temperature is about or equal to 60° C. In certain embodiments, the optimized reaction time is about or equal to 60 minutes.

In certain embodiments of any of the methods provided herein, the step of determining whether the biological sample comprises T. tenax, when performed using LAMP, is at least 100-times more sensitive than conventional PCR.

In embodiments of any of the methods provided herein, the step of determining whether the biological sample comprises T. tenax does not detect any one or more of the microbes comprising T. vaginalis, S. pyogenes, S. aureus, E. coli, E. faecalis, and C. albicans.

In embodiments of any of the methods provided herein, when the step of determining whether the biological sample comprises T. tenax comprises performing LAMP analysis of the nucleic acid (e.g., DNA or RNA) in the biological sample, the step of performing LAMP comprises use of the primers of SEQ ID NOS:2-7, as listed in TABLE 1. In embodiments of any of the methods provided herein, when the step of determining whether the biological sample comprises T. tenax comprises performing LAMP analysis of the nucleic acid (e.g., DNA or RNA) in the biological sample, the step of performing LAMP comprises use of the primers of SEQ ID NOS:2-7, as listed in TABLE 1.

In embodiments of any of the methods provided herein, when the step of determining whether the biological sample comprises T. tenax comprises performing analysis of the nucleic acid (e.g., DNA or RNA) in the biological sample by amplification methods using primer pairs, such as by PCR or RT-PCR (reverse transcription PCR), the step of performing the amplification comprises use of the primer pair comprising SEQ ID NO:6 and SEQ ID NO:7, as listed in TABLE 1.

Provided herein are methods for analyzing nucleic acid from a biological sample, comprising:
contacting the biological sample, or nucleic acid obtained from the biological sample, with at least one polynucleotide primer pair under amplification conditions, thereby preparing a mixture; and analyzing nucleic acid of the mixture; wherein: the polynucleotide primer pair(s) hybridizes to subsequences of SEQ ID NO:1, if present in the nucleic acid of the biological sample, under the amplification conditions.

Also provided herein are methods for preparing a nucleic acid mixture, comprising: contacting a biological sample, or nucleic acid of a biological sample, with at least one polynucleotide primer pair under amplification conditions, thereby preparing a mixture, wherein: the polynucleotide primer pair(s) hybridizes to subsequences of SEQ ID NO:1, if present in the nucleic acid of the biological sample, under the amplification conditions. In certain embodiments, the methods further comprise analyzing the nucleic acid of the mixture.

Also provided herein are methods for analyzing nucleic acid from a biological sample, comprising: contacting the biological sample, or nucleic acid obtained from the biological sample, with at least one polynucleotide primer pair under amplification conditions, thereby generating one or more amplification products; and analyzing the amplification products, wherein:

the polynucleotide primer pair(s) hybridizes to subsequences of SEQ ID NO:1, if present in the nucleic acid of the biological sample, under the amplification conditions.

Also provided herein are methods for generating nucleic acid amplification products from a biological sample, comprising: contacting the biological sample, or nucleic acid obtained from the biological sample, with at least one polynucleotide primer pair under amplification conditions, thereby generating one or more amplification products, wherein: the polynucleotide primer pair(s) hybridizes to subsequences of SEQ ID NO:1, if present in the nucleic acid of the biological sample, under the amplification conditions. In certain embodiments, the methods further comprise analyzing the amplification products.

In embodiments of any of the methods provided herein, analyzing the nucleic acid, or amplification products generated under amplification conditions, comprises determining the presence or absence of T. tenax nucleic acid in the biological sample. In certain embodiments, if T. tenax nucleic acid is determined to be present in the biological sample, analyzing the nucleic acid, or amplification products generated under amplification conditions, further comprises quantitating the amount of T. tenax nucleic acid in the biological sample based on the amount of amplification products generated under amplification conditions.

In embodiments of any of the methods provided herein, one or both primers of the at least one primer pair is selected from among a sequence that is identical, or substantially identical, to: a sequence of SEQ ID NO:1, or to a subsequence of SEQ ID NO:1, or to a complement of SEQ ID NO:1, or to a complement of a subsequence of SEQ ID NO:1. In certain embodiments, at least one polynucleotide of the at least one primer pair is identical, or substantially identical, to a subsequence of SEQ ID NO:1, or to a complement of a subsequence of SEQ ID NO:1, where the subsequence of SEQ ID NO:1 comprises an ITS region of T. tenax or a portion thereof, the 5.8S rRNA gene of T. tenax or a portion thereof, or any combination of regions or portions thereof comprising an ITS region and/or the 5.8S rRNA gene of T. tenax. In embodiments, the at least one polynucleotide primer pair comprises a forward primer and a reverse primer. In some embodiments, the at least one polynucleotide primer pair comprises the sequences set forth in SEQ ID NO:6 and SEQ ID NO:7.

In embodiments of any of the methods provided herein, the nucleic acid can be analyzed using a single primer pair under amplification conditions (e.g., PCR, or qPCR). In certain embodiments, the methods provided herein comprise contacting nucleic acid of a plant sample with a plurality of polynucleotide primer pairs under amplification conditions. In some embodiments, the method comprises contacting the nucleic acid of the biological sample with a set of loop mediated isothermal amplification (LAMP) primers under the amplification conditions. In certain embodiments, the LAMP primers comprise a set of primers comprising the polynucleotides of SEQ ID NO:2 to SEQ ID NO:7.

In embodiments of any of the methods provided herein, based on analyzing the nucleic acid, or analyzing amplification products of the nucleic acid, the presence or absence of T. tenax in the biological sample is determined. In certain embodiments, analyzing the nucleic acid, or the amplification products of the nucleic acid, in the biological sample further comprises quantitating the nucleic acid, or the amplification products of the nucleic acid. In some embodiments, based on quantitating the nucleic acid, or the amplification products of the nucleic acid, the amount of T. tenax in the biological sample is quantified.

Also provided herein are methods for determining the presence, absence and/or amount of T. tenax in a biological sample, comprising: contacting the biological sample, or nucleic acid from the biological sample, with at least one polynucleotide primer pair under amplification conditions and amplifying the sample, thereby preparing an amplified nucleic acid mixture, wherein, if T. tenax is present, at least one polynucleotide primer pair is capable of specifically hybridizing to a subsequence of the nucleic acid of T. tenax, or to a complement thereof; determining the presence, absence and/or amount of at least one amplicon that is an amplification product of a polynucleotide primer pair in the amplified nucleic acid mixture, thereby determining the presence, absence and/or amount of T. tenax in the biological sample. In certain embodiments, at least one polynucleotide of the at least one primer pair is identical, or substantially identical, to a subsequence of SEQ ID NO:1, or to a complement of a subsequence of SEQ ID NO:1, where the subsequence of SEQ ID NO:1 comprises an ITS region of T. tenax or a portion thereof, the 5.8S rRNA gene of T. tenax or a portion thereof, or any combination of regions or portions thereof comprising an ITS region and/or the 5.8S rRNA gene of T. tenax. In embodiments, the at least one polynucleotide primer pair comprises a forward primer and a reverse primer. In some embodiments, the at least one polynucleotide primer pair comprises the sequences set forth in SEQ ID NO:6 and SEQ ID NO:7.

Also provided herein are methods of detecting the presence or absence of T. tenax in a biological sample comprising contacting the biological sample, or nucleic acid of the biological sample, with a set of loop-mediated isothermal amplification (LAMP) primers under amplification conditions and analyzing the resulting amplification products whereby, based on analyzing the amplification products, the presence or absence of T. tenax in the biological sample is determined. In certain embodiments, at least one amplicon that is an amplification product in the amplified nucleic acid mixture is obtained by amplification of a subsequence of SEQ ID NO:1, or a complement of a subsequence of SEQ ID NO:1. In embodiments, the subsequence of SEQ ID NO:1 comprises an ITS region of T. tenax or a portion thereof, the 5.8S rRNA gene of T. tenax or a portion thereof, or any combination of regions or portions thereof comprising an ITS region and/or the 5.8S rRNA gene of T. tenax. In certain embodiments, the set of loop-mediated isothermal amplification (LAMP) primers comprises the sequences set forth in SEQ ID NO:2 to SEQ ID NO:7.

In embodiments of any of the methods provided herein, at least one amplicon that is an amplification product in the amplified nucleic acid mixture is obtained by amplification of a subsequence of T. tenax genomic DNA, or a complement thereof. In certain embodiments of any of the methods provided herein, at least one amplicon that is an amplification product in the amplified nucleic acid mixture is obtained by amplification of a subsequence of T. tenax RNA, or cDNA, or a complement thereof.

In embodiments of any of the methods provided herein, the subsequence, a portion thereof or a complement or portion thereof of T. tenax nucleic acid that is amplified is non-identical to any subsequence of the nucleic acids of other protozoan parasites or microbes/pathogens. In embodiments, the other protozoan parasites or microbes/pathogens are selected from among one or more of T. vaginalis, S. pyogenes, S. aureus, E. coli, E. faecalis, and C. albicans. In certain embodiments of any of the methods provided herein, the methods specifically detect T. tenax nucleic acid and do not detect other protozoan parasites or microbes/pathogens. In embodiments, the methods specifically detect T. tenax nucleic acid and do not detect other protozoan parasites or microbes/pathogens selected from among one or more of T. vaginalis, S. pyogenes, S. aureus, E. coli, E. faecalis, and C. albicans.

In certain embodiments of any of the methods provided herein, the amplification conditions for performing LAMP comprises a Mg²⁺ salt, such as MgSO₄, whereby the Mg²⁺ concentration (mM) is about 3 mM to about 12 mM, such as about 4 mM to about 10 mM, or about 5 mM to about 9 mM, or about 5 mM to about 7 mM, or about or equal to 3 mM, 4 mM, 5 mM, 6 mM, 7 mM, 8 mM, 9 mM, 10 mM, 11 mM or 12 mM. In embodiments, the amplification conditions for performing LAMP comprises a Mg²⁺ concentration of about or equal to 6 mM.

In certain embodiments of any of the methods provided herein, the amplification conditions for performing LAMP comprises a temperature of about 55° C. to about 65° C., such as about 56° C. to about 64° C., or about 57° C. to about 63° C., or about 58° C. to about 62° C., or about 59° C. to about 61° C., or about or equal to 55° C., 56° C., 57° C., 58° C., 59° C., 60° C., 61° C., 62° C., 63° C., 64° C. or 65° C. In embodiments, the amplification conditions for performing LAMP comprises a temperature of about or equal to 60° C.

In certain embodiments of any of the methods provided herein, the amplification conditions for performing LAMP comprises a reaction time (minutes) of about 25 minutes to about 90 minutes, such as about 30 minutes to about 85 minutes, or about 35 minutes to about 80 minutes, or about 40 minutes to about 75 minutes, or about 45 minutes to about 70 minutes, or about 55 minutes to about 65 minutes or about or equal to 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 54 minutes, 55 minutes, 56 minutes, 57 minutes, 58 minutes, 59 minutes, 60 minutes, 61 minutes, 62 minutes, 63 minutes, 64 minutes or 65 minutes. In embodiments, the amplification conditions for performing LAMP comprises a reaction time of 10 about or equal to 60 minutes.

Also provided herein are methods of detecting the presence or absence of T. tenax in a biological sample comprising contacting the biological sample, or nucleic acid of the biological sample, with a set of loop-mediated isothermal amplification (LAMP) primers under amplification conditions, wherein the set of loop-mediated isothermal amplification (LAMP) primers comprises the sequences set forth in SEQ ID NO:2 to SEQ ID NO:7 and the amplification conditions comprise a MgSO₄ concentration of about or equal to 6 mM, a temperature of about or equal to 60° C. and a reaction time of about or equal to 60 minutes, and analyzing the resulting amplification products whereby, based on analyzing the amplification products, the presence or absence of T. tenax in the biological sample is determined.

In embodiments of any of the methods provided herein, analysis of the nucleic acid and/or the amplification products derived therefrom can comprise detecting the presence or absence of T. tenax nucleic acid in a biological sample and, further, if T. tenax is identified as being present in the biological sample, quantitating the T. tenax nucleic acid and/or the amplification products derived therefrom. Quantitation of nucleic acid can be performed by methods known to those of skill in the art including, but not limited to, quantitative PCR (qPCR), reverse transcriptase qPCR (RT-qPCR) and quantitative LAMP methods.

The biological samples used in the methods provided herein are obtained from a subject or previously have been obtained from a subject. The subject, in certain embodiments, can be any eukaryotic species including, but not limited to, vertebrate, mammalian, human and other primates, porcine, bovine, feline, avian, equine, canine, and other eukaryotic species. In embodiments, the subject is a human. In certain embodiments, the subject is a canine.

Biological samples that can be analyzed according to any embodiments of the methods provided herein include, but are not limited to, blood, plasma, serum, urine, saliva, seminal fluid, lavages (e.g., bronchoalveolar, gastric, peritoneal, ductal, ear, arthroscopic), cervical fluid, cervicovaginal fluid, cerebrospinal fluid, vaginal fluid, breast fluid, breast milk, synovial fluid, semen, seminal fluid, sputum, cerebral spinal fluid, tears, mucus, interstitial fluid, follicular fluid, amniotic fluid, aqueous humor, vitreous humor, peritoneal fluid, ascites, sweat, lymphatic fluid, lung sputum or fractions or components thereof. In certain embodiments, the biological sample comprises an oral swab or saliva or urine.

Also provided herein are methods of selecting a subject for potential treatment of a disease associated with the presence of T. tenax, comprising, detecting the presence or absence of T. tenax in a biological sample from a subject according to any of the methods provided herein and, if T. tenax is detected in the biological sample, selecting the subject for potential treatment of the disease. In embodiments, the methods further comprise identifying the subject as having a disease associated with the presence of T. tenax and/or treating the disease. In certain embodiments, the disease is a periodontal disease. In some embodiments, the disease is a sexually transmitted disease.

Provided herein are compositions for performing any of the methods provided herein. For example, provided herein are compositions comprising one or more polynucleotide primer pairs wherein: one or both primers of the at least one primer pair is selected from among a sequence that is identical, or substantially identical, to: a sequence of SEQ ID NO:1, or to a subsequence of SEQ ID NO:1, or to a complement of SEQ ID NO:1, or to a complement of a subsequence of SEQ ID NO:1. In certain embodiments, at least one polynucleotide primer of the at least one primer pair is identical, or substantially identical, to a subsequence of SEQ ID NO:1, or to a complement of a subsequence of SEQ ID NO:1, where the subsequence of SEQ ID NO:1 comprises an ITS region of T. tenax or a portion thereof, the 5.8S rRNA gene of T. tenax or a portion thereof, or any combination of regions or portions thereof comprising an ITS region and/or the 5.8S rRNA gene of T. tenax. In embodiments, the at least one polynucleotide primer pair comprises a forward primer and a reverse primer. In some embodiments, the at least one polynucleotide primer pair comprises the sequences set forth in SEQ ID NO:6 and SEQ ID NO:7. In embodiments, the compositions further comprise reagents for replicating and/or amplifying a target nucleic acid, e.g., reagents for performing PCR or qPCR.

Also provided herein are compositions comprising a set of loop-mediated isothermal amplification (LAMP) primers, wherein the primers of at least one primer pair are selected from among a sequence that is identical, or substantially identical, to: a sequence of SEQ ID NO:1, or to a subsequence of SEQ ID NO:1, or to a complement of SEQ ID NO:1, or to a complement of a subsequence of SEQ ID NO:1. In certain embodiments, at least one polynucleotide of the at least one primer pair is identical, or substantially identical, to a subsequence of SEQ ID NO:1, or to a complement of a subsequence of SEQ ID NO:1, where the subsequence of SEQ ID NO:1 comprises an ITS region of T. tenax or a portion thereof, the 5.8S rRNA gene of T. tenax or a portion thereof, or any combination of regions or portions thereof comprising an ITS region and/or the 5.8S rRNA gene of T. tenax. In certain embodiments, the set of loop-mediated isothermal amplification

(LAMP) primers in the compositions provided herein comprises the sequences set forth in SEQ ID NO:2 to SEQ ID NO:7. In embodiments, the compositions further comprise reagents for performing LAMP analysis of a target nucleic acid, such as a buffer and a magnesium salt. In certain embodiments, the magnesium salt is MgSO₄.

Also provided herein are isolated polynucleotides comprising or one or more polynucleotides for detecting T. tenax, wherein the polynucleotide sequences are identical, or substantially identical, to a sequence of SEQ ID NO:1, or to a subsequence of SEQ ID NO:1, or to a complement of SEQ ID NO:1, or to a complement of a subsequence of SEQ ID NO:1. In certain embodiments, the sequences of the isolated polynucleotides are identical, or substantially identical, to a subsequence of SEQ ID NO:1, or to a complement of a subsequence of SEQ ID NO:1, where the subsequence of SEQ ID NO:1 comprises an ITS region of T. tenax or a portion thereof, the 5.8S rRNA gene of T. tenax or a portion thereof, or any combination of regions or portions thereof comprising an ITS region and/or the 5.8S rRNA gene of T. tenax. In embodiments, the isolated polynucleotides comprise the sequences set forth in SEQ ID NO:2 to SEQ ID NO:7.

Also provided herein are kits comprising any of the compositions or isolated polynucleotides provided herein, and instructions for use in analyzing T. tenax nucleic acid.

### BRIEF DESCRIPTION OF THE DRAWINGS

For the purpose of illustrating the disclosure, depicted in the drawings are certain embodiments of the disclosure. However, the disclosure is not limited to the precise arrangements and instrumentalities of the embodiments depicted in the drawings.

FIG. 1A depicts the temperature optimization of the LAMP reaction.

FIG. 1B depicts the time optimization of the LAMP reaction.

FIG. 2A depicts the analytical sensitivity of the LAMP assay.

FIG. 2B depicts the analytical sensitivity of conventional PCR.

FIG. 3 depicts the analytical specificity of the LAMP assay to T. tenax.

FIG. 4 depicts the LAMP assay performed on serially diluted samples of T. tenax in PBS or TE buffer.

FIG. 5A depicts the results of the LAMP assay performed on T. tenax spiked canine saliva following boiling at 100° C. for 30 min without DNA extraction.

FIG. 5B depicts the results of PCR performed on T. tenax spiked canine saliva following boiling at 100° C. for 30 min without DNA extraction.

FIG. 5C depicts the results of the LAMP assay performed on washed T. tenax spiked canine saliva followed by resuspension in Tris-EDTA (TE) buffer without DNA extraction.

FIG. 6 depicts the results of the LAMP assay performed on canine oral swabs without DNA extraction.

FIG. 7 depicts the results of the LAMP assay performed on canine oral swabs without DNA extraction.

### DETAILED DESCRIPTION

Trichomonas tenax (T. tenax) is a flagellated protozoan that inhabits the human and canine oral cavity in patients with poor oral hygiene and periodontal disease. The protozoan belongs to the same genus as T. vaginalis, a human pathogen of great medical importance worldwide. In addition, T. tenax has also been detected in other tissues and organs of the body such as the respiratory tract, the urogenital tract, and lymph nodes. T. tenax has been reported in humans since the 1960s and has been specifically connected to human periodontal disease. In a prospective study carried out in Jordan recently, it was found that the prevalence of T. tenax among health, gingivitis and periodontitis groups is 3.2%, 5.7% and 25.6%, respectively. In the periodontitis group, the prevalence of T. tenax among individuals with generalized or localized conditions is 19.6% and 0.0%, respectively (p=0.039). Furthermore, the odds ratios with T. tenax infection between periodontitis and healthy groups, and between periodontitis and gingivitis groups are 4.7 (95% CI: 1.0-21.8, p=0.045) and 7.2 (95% CI: 1.5-33.8, p=0.013), respectively. In a study of 92 samples of canine plaque using touchdown PCR and next generation sequencing, the proportion of Trichomonas species among healthy, gingivitis, early and severe periodontitis is 3.5%, 2.8%, 6.1% and 35.0%, respectively.

Despite its relatively frequent association with periodontal diseases, T. tenax has not received enough attention as a zoonotic pathogen, mainly due to the lack of simple detection methods. Over the past years, researchers have been using PCR to detect and identify T. tenax, and update the prevalence of this parasite from traditional diagnostic methods previously used which included culture and microscopy. PCR has a higher sensitivity compared to culture and microscopy. Despite these advantages, PCR assays still require expensive laboratory equipment, reagents, highly trained professionals and can be time-consuming to achieve accurate results. Further, PCR does not often distinguish T. tenax from the closely related human parasite T. vaginalis without DNA sequencing. T. tenax and T. vaginalis mainly locate in the mouth cavity and the urogenital tract, respectively. Lately, T. tenax has been unequivocally detected in human urogenital tract as well by sequencing PCR products. Therefore, T. tenax, along with T. vaginalis, contributes to the most common sexually transmitted diseases caused by microbes other than viruses among humans.

Provided herein are methods for analyzing nucleic acid from a biological sample for the presence, absence or amount of nucleic acid from a protozoan parasite, such as T. tenax. Also provided herein are methods for generating nucleic acid amplification products from a biological sample containing a protozoan parasite, such as T. tenax. Also provided herein are methods for preparing a nucleic acid mixture from a biological sample containing a protozoan parasite, such as T. tenax. In certain embodiments, the methods provided herein determine the presence, absence and/or amount of a protozoan parasite, such as T. tenax, in the biological sample. A method herein may comprise contacting nucleic acid of a biological sample with a polynucleotide primer pair under amplification conditions, wherein the polynucleotide primer pair specifically hybridizes to a subsequence of T. tenax nucleic acid, such as genomic DNA, or RNA, or cDNA derived therefrom. In some embodiments, a method herein comprises contacting nucleic acid of a biological sample with one or more polynucleotide primer pairs under amplification conditions. In some embodiments, a method herein comprises contacting nucleic acid of a biological sample with a plurality of polynucleotide primer pairs under amplification conditions. A plurality of primer pairs may comprise two or more polynucleotide primer pairs, three or more polynucleotide primer pairs, four or more polynucleotide primer pairs, five or more polynucleotide primer pairs, six or more polynucleotide primer pairs, or more.

In some embodiments, a method comprises generating one or more amplification products. Amplification products may be generated by any suitable amplification method described herein or known in the art (e.g., polymerase chain reaction (PCR)). Suitable amplification conditions include any conditions that can generate an amplification product, when a target nucleic acid is contacted with primers that are capable of hybridizing to the target nucleic acid. In some embodiments, a method comprises generating a mixture (e.g., a mixture of two or more amplification product species). A mixture of two or more amplification product species may be generated when two or more primer pairs hybridize to different regions of a target nucleic acid. Such amplification product species may have different lengths and/or different nucleotide sequences, which may include overlapping and/or non-overlapping sequences.

Generally, a primer pair comprises a forward primer and a reverse primer. Two primer pairs may comprise two different forward primer species (e.g., A-fwd and B-fwd) and two different reverse primer species (e.g., A-rev, B-rev); may comprise one forward primer species (e.g., A-fwd) and two different reverse primer species (e.g., A-rev, B-rev); or may comprise two different forward primer species (e.g., A-fwd and B-fwd) and one reverse primer species (e.g., A-rev), provided the combination of forward and reverse primer species is capable of generating two amplification product species. Further forward and reverse primer combinations are contemplated for additional primer pairs. For T. tenax, examples of forward and reverse primer pairing combinations, such as the F3 and B3 primer pair, are provided in TABLE 1 herein.

Provided herein are methods and compositions for determining the presence, absence and/or amount of a protozoan parasite, such as T. tenax, in a subject, which include: (a) obtaining a nucleic acid sample from a biological sample; (b) contacting the nucleic acid sample with at least one polynucleotide primer pair under amplification conditions and amplifying the sample, thereby preparing an amplified nucleic acid mixture, wherein, if the protozoan parasite is present, the polynucleotide primer pair is capable of specifically hybridizing to and amplifying a subsequence of the nucleic acid of the protozoan parasite, or a complement thereof, thereby determining the presence, absence and/or amount of the protozoan parasite.

In the methods and compositions provided herein, in embodiments, the polynucleotide primer pairs for specifically hybridizing to and amplifying a subsequence of the nucleic acid of the protozoan parasite, such as T. tenax, are designed to amplify a subsequence that is non-identical to any subsequence of the nucleic acids of other protozoan parasites or microbes/pathogens, thereby permitting specific detection of, e.g., T. tenax, and avoiding non-specific detection of sequences of other protozoan parasites or microbes/pathogens, or the host (subject, e.g., human or canine subject)) from which a biological sample for analyzing the presence or absence of nucleic acid from T. tenax is obtained. A subsequence that is non-identical to another subsequence, or complement thereof, generally refers to a subsequence containing one or more mismatched nucleotides when aligned with consecutive bases of another subsequence of equivalent length (e.g., identical length, a length that is about 95%, about 96%, about 97%, about 98%, about 99%, about 100%, about 101%, about 102%, about 103%, about 104% or about 105% of the length of the subsequence to which it is compared, or a length that is longer or shorter by one nucleotide, two nucleotides, or three nucleotides than the subsequence to which it is compared). In certain embodiments, the length of the sequence to which the subsequence of equivalent length is compared is about 15 nucleotides to about 30 nucleotides, or a length that is about 95%, about 96%, about 97%, about 98%, about 99%, about 100%, about 101%, about 102%, about 103%, about 104% or about 105% of a sequence of length between about 15 nucleotides to about 30 nucleotides.

Primer sequences and length may affect hybridization to target nucleic acid sequences. Depending on the degree of mismatch between the primer and target nucleic acid, low, medium or high stringency conditions may be used to effect primer/target annealing. As used herein, the term “stringent conditions” refers to conditions for hybridization and washing. Methods for hybridization reaction temperature condition optimization are known to those of skill in the art and may be found, for example, in Current Protocols in Molecular Biology, John Wiley & Sons, N.Y., 6.3.1-6.3.6 (1989); either aqueous or non-aqueous methods are described in that reference and either can be used. Non-limiting examples of stringent hybridization conditions are hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5 M sodium phosphate, 7% SDS at 65° C., followed by one or more washes at 0.2×SSC, 1% SDS at 65° C. Stringent hybridization temperatures can also be altered (i.e., lowered) with the addition of certain organic solvents, formamide for example. Organic solvents, like formamide, reduce the thermal stability of double-stranded polynucleotides, so that hybridization can be performed at lower temperatures, while still maintaining stringent conditions and extending the useful life of nucleic acids that may be heat labile. As used herein: stringency of hybridization in determining percentage mismatch are those conditions understood by those of skill in the art and typically are substantially equivalent to the following: 1) high stringency: 0.1×SSPE, 0.1% SDS, 65° C.; 2) medium stringency: 0.2×SSPE, 0.1% SDS, 50° C.; 3) low stringency: 1.0×SSPE, 0.1% SDS, 50° C. It is understood that equivalent stringencies may be achieved using alternative buffers, salts and temperatures.

The terms “specifically hybridizes,” “specific hybridization” and the like, as used herein, refers to conditions under which a polynucleotide primer pair preferentially hybridizes to a particular subsequence, e.g., of the nucleic acid of a protozoan parasite or other microbe/pathogen, such as T. tenax, and hybridizes to a substantially lesser degree, e.g., 5% or less, such as 5%, 4%, 3%, 2%, 1% or 0%, or between 0% to 1%, 2%, 3%, 4% or 5% or less, to any other subsequence of the nucleic acid of the protozoan parasite or other microbe/pathogen, or to subsequences of the nucleic acid of any other protozoan parasites or other microbes/pathogens, or to subsequences of the nucleic acid of the subject from which the biological sample is derived. In embodiments, the specific hybridization is under conditions of high stringency, or under conditions of medium stringency.

Nucleic acid may be derived from one or more biological samples by methods known in the art. Any suitable method can be used for isolating, extracting and/or purifying DNA from a biological sample, non-limiting examples of which include methods of DNA preparation (e.g., described by Sambrook and Russell, Molecular Cloning: A Laboratory Manual 3d ed., 2001), various commercially available reagents or kits, such as DNeasy®, RNeasy®, QIAprep®, QIAquick®, and QIAamp®, nucleic acid isolation/purification kits by Qiagen, Inc. (Germantown, Md); DNAzol®, ChargeSwitch®, Purelink®, GeneCatcher® nucleic acid isolation/purification kits by Life Technologies, Inc. (Carlsbad, CA); NucleoMag®, NucleoSpin®, and NucleoBond® nucleic acid isolation/purification kits by Clontech Laboratories, Inc. (Mountain View, CA), DNA/RNA extraction kits from Zymo Research (e.g., ZYMOBIOMICS DNA Mini Kit, ZYMOBIOMICS DNA/RNA Miniprep Kit, ZYMOCLEAN gel DNA recovery); the like or combinations thereof.

Nucleic acid may be provided for performing methods described herein with or without processing of the sample(s) containing the nucleic acid. In some embodiments, nucleic acid is provided for conducting methods described herein after processing of the sample(s) containing the nucleic acid. For example, a nucleic acid can be extracted, isolated, purified, partially purified and/or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” as used herein can refer to a nucleic acid removed from a test subject (e.g., a human or a canine). An isolated nucleic acid can be provided with fewer non-nucleic acid components (e.g., protein, lipid) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components. The term “purified” as used herein can refer to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components. The term “purified” as used herein can refer to a nucleic acid provided that contains fewer nucleic acid species than in the sample source from which the nucleic acid is derived. A composition comprising purified nucleic acid may be about 90%, 91%, 92%, 93%, 94%, 95%, 96%, 97%, 98%, 99% or greater than 99% free of other nucleic acid species. In certain examples, nucleic acid can be purified from a mixture comprising protozoan parasite/microbe/pathogen and host (e.g., the host is a human or canine subject) nucleic acid. In some embodiments, nucleic acid is provided for performing methods described herein without prior processing of the sample(s) containing the nucleic acid. For example, nucleic acid may be analyzed directly from a sample without prior extraction, purification, partial purification, and/or amplification.

There is a need for quick, cheap, easy and reliable methods for detection of T. tenax. Over the last two decades, a method termed loop-mediated isothermal amplification (LAMP) has established itself as a method that amplifies DNA with high specificity, efficiency, and rapidity under isothermal conditions. LAMP uses a DNA polymerase with strand displacement activity and a set of 4-6 primers that amplifies DNA under isothermal conditions; thus, incubation can be done in a heat block or water bath. Studies have shown that LAMP can be a rapid, cost-effective, sensitive and specific nucleic acid amplification test that can be used for the diagnosis of various infectious diseases especially in developing countries. For example, it has been used to detect protozoa such as Toxoplasma sp., Trypanosoma sp., Cryptosporidium sp., Entamoeba histolytica, Tritrichomonas foetus, and T. vaginalis; and bacteria such as Mycobacterium tuberculosis, Yersinia enterocolitica, and Enterococcus faecalis.

In some embodiments of the method provided herein, an amplification method comprises loop mediated isothermal amplification (LAMP). Loop-mediated isothermal amplification (LAMP) is a single-tube technique useful for nucleic acid amplification. Reverse transcription loop-mediated isothermal amplification (RT-LAMP) combines LAMP with a reverse transcription step for the detection of RNA. LAMP is typically performed under isothermal conditions. In contrast to a polymerase chain reaction (PCR) technology, which is typically performed using a series of alternating temperature cycles, isothermal amplification is performed at a constant temperature, and does not require a thermal cycler.

In LAMP, a target sequence is amplified at a constant temperature (e.g., between about 60° C. to about 65° C.) using a plurality of primer pairs (e.g., two primer pairs, three primer pairs) and a polymerase (e.g., a polymerase with high strand displacement activity). In certain applications, four different primers may be used to amplify six distinct regions on a target sequence, for example, which may increase specificity. An additional pair of loop primers can further accelerate the reaction.

The amplification product can be detected via photometry (i.e., measuring the turbidity caused by magnesium pyrophosphate precipitate in solution as a byproduct of amplification). This generally allows for visualization by the naked eye or by photometric detection approaches (e.g., for small volumes). In certain applications, the reaction can be followed in real-time either by measuring turbidity or by fluorescence using intercalating dyes (e.g., SYTO 9, SYBR green). Certain dyes may be used to create a visible color change that can be seen with the naked eye without the need for specialized equipment. Dye molecules intercalate or directly label the DNA, and in turn can be correlated with the number of copies initially present. Accordingly, certain variations of LAMP may be quantitative. Detection of LAMP amplification products also may be achieved using manganese loaded calcein, which starts fluorescing upon complexation of manganese by pyrophosphate during in vitro DNA synthesis. Another method for visual detection of LAMP amplification products by the naked eye is based on the ability of the products to hybridize with complementary gold-bound single-stranded DNA, which prevents a red to purple-blue color change that would otherwise occur during salt-induced aggregation of the gold particles.

A number of LAMP visualization technologies are known to those of skill in the art (see, e.g., Fischbach et al., Biotechniques, 58(4):189-194 (2015), the contents of which are incorporated in their entirety by reference herein). Examples of such visualization reagents, summarized in the Table below from Fischbach et al., include magnesium pyrophosphate, hydroxynaphthol blue (HNB), calcein, SYBR Green I, EvaGreen and the nucleic acid-specific dye, berberine, which emits a fluorescent signal under UV light after a positive LAMP reaction:

| | | Hydroxynaphthol | | | | |
|---|---|---|---|---|---|---|
| | Turbidity | blue | Calcein | SYBR Green I | EvaGreen | Berberine |
| Substance | Mg-pyrophosphate | Hydroxynaphthol | Calcein AM + | SYBR Green I | EvaGreen | Berberine-SO₄ |
| | (Mg-PPi) | blue (HNB) | MnCl₂ | | | |
| Origin | Amplification | Synthetic | Synthetic | Synthetic | Synthetic | Natural |
| | product | | | | | |
| Toxicity | None | May cause eye | May be | Mutation | Possible | May be toxic in |
| | | irritation | harmful to skin | enhancer | carcinogen | high |
| | | | and eyes | | | concentrations |
| Detection | Insoluble complex; | Decrease of free | Decrease of | dsDNA | dsDNA | Small groove |
| Mechanism | precipitation | Mg²⁺ | free Mn²⁺ | intercalation | intercalation | intercalation |
| Readout | Turbidity | Absorption | Fluorescence | Fluorescence | Fluorescence | Fluorescence |
| | Absorbance: | Absorbance: | Excitation: 495 | Excitation: 494 | Excitation: 500 | Excitation: 450 |
| | 400 nm | 650 nm | nm Emission: | nm Emission: | nm Emission: | nm Emission: |
| | | | 515 nm | 521 nm | 530 nm | 530 nm |
| Effect on | None | None | Manganese | Not inhibiting | Not inhibiting | Not inhibiting |
| amplification | | | may inhibit | when used 0.5- | when used 0.5- | (≤180 μM) |
| | | | reaction | 1x | 1x | |
| One-pot real- | + + | − | + + + | + + + + | + + + + | + + + + |
| time assay | | | | | | |
| Equipment | Turbidometer | − | Fluorometer | Fluorometer | Fluorometer | Fluorometer |
| for | | | with | with | with | with |
| real-time | | | FAM filter | FAM filter | FAM filter | FAM filter |
| detection | | | | | | |
| One-pot end | + + + | + + + + | + + + + | − | − | + + + |
| point assay | | | | | | (UV light with |
| | | | | | | low background |
| | | | | | | signal) |
| Equipment | None (cordless | None | UV lamp | Not applicable | Not applicable | UV lamp |
| for | centrifuge) | | (optional) | | | |
| end point | | | | | | |
| detection | | | | | | |
| Evaluation of | +Turbid | +Sky blue | +Green | +Fluorescence | +Fluorescence | +Fluorescence |
| results | −Clear | −Violet | −Orange | signal | signal | signal |
| | | | | −No signal | −No signal | −No signal |
| Percentage of | 0 | <0.1 | <0.1 | 26.01 | 1.18 | 0.82 |
| overall costs * | | | | | | |
| Relative | + + + | + + + | + + + | + + + | + + + | + + + |
| sensitivity ** | | | | | | |
| Field | + + + | + + + + | + + + + | + + | + + | + + + + |
| applicability *** | | | | | | |

Summary of features relevant for in-the-field loop-mediated isothermal amplification (LAMP) assays tested for detection of potato spindle tuber viroid (PSTVd).
Number of “+” describes the applicability/relevance of the feature.
* Overall costs represent the basic chemicals of common suppliers for one LAMP reaction, depending on reaction volume.
** In our assays. Analytical sensitivity may depend on LAMP setup and has to be optimized separately.
*** Depending on visualization.

The instant subject matter is directed to a method for detecting protozoan parasites, such as T. tenax, and other microbes/pathogens, using amplification methods. In embodiments, the methods provided herein can detect T. tenax by amplification, e.g., PCR amplification, using forward and reverse primer pairs, e.g., the F3 and B3 primer pair set forth in TABLE 1. In embodiments, the methods provided herein are for detecting T. tenax via LAMP assay. In certain embodiments, the methods provided herein are for detecting T. tenax via LAMP assay using a primer set of 6 primers comprising the polynucleotides of SEQ ID NO:2 to SEQ ID NO:7. In embodiments of any of the methods provided herein, in certain embodiments, each polynucleotide of one or more primer pairs for detecting T. tenax by amplification, or each polynucleotide of one or more primer pair sets for detecting T. tenax by LAMP, is identical, or substantially identical, to a sequence of SEQ ID NO:1, or to a subsequence of SEQ ID NO:1, or to a complement of SEQ ID NO:1, or to a complement of a subsequence of SEQ ID NO:1. In certain embodiments, the methods provided herein can include one or more polynucleotides of one or more primer pairs for detecting T. tenax by amplification, or one or more polynucleotides of one or more primer pair sets for detecting T. tenax by LAMP, which are identical, or substantially identical, to a sequence of SEQ ID NO:1, or to a subsequence of SEQ ID NO:1, or to a complement of SEQ ID NO:1, or to a complement of a subsequence of SEQ ID NO:1. The term “substantially identical,” as used herein, refers to a consecutive sequence of nucleotides that is about 90% to about 99% or more, such as at least or about 90%, at least or about 91%, at least or about 92%, at least or about 93%, at least or about 94%, at least or about 95%, at least or about 96%, at least or about 97%, at least or about 98%, or at least or about 99% or more identical to a consecutive sequence of nucleotides of a sequence of SEQ ID NO:1, or of a subsequence of SEQ ID NO:1, or of a complement of SEQ ID NO:1, or of a complement of a subsequence of SEQ ID NO:1. The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=# of identical positions/total # of positions×100). When a position in one sequence is occupied by the same nucleotide as the corresponding position in the other sequence, then the molecules are identical at that position.

In embodiments of the methods provided herein, each polynucleotide of one or more primer pairs for detecting T. tenax by amplification, or at least one polynucleotide of a primer pair for detecting T. tenax by amplification, or each polynucleotide of one or more primer pair sets for detecting T. tenax by LAMP, or at least one polynucleotide of a primer pair set for detecting T. tenax by LAMP, is identical, or substantially identical, to a subsequence of SEQ ID NO:1, or to a complement of a subsequence of SEQ ID NO:1, where the subsequence of SEQ ID NO:1 comprises an ITS region of T. tenax or a portion thereof, the 5.8S rRNA gene of T. tenax or a portion thereof, or any combination of regions or portions thereof comprising an ITS region and/or the 5.8S rRNA gene of T. tenax. The LAMP assay provided herein has high sensitivity and specificity and provides a quick, cheap, easy and reliable method used for the detection of T. tenax that can be used as a point-of-care test in human and veterinary medicine.

The currently disclosed LAMP assay for detection of T. tenax is highly sensitive. In some embodiments, the LAMP assay is at least 100-times more sensitive than conventional PCR. By way of example but not limitation, the sensitivity of the LAMP assay may be 100-times, 250-times, 500-times, 750-times, 1000-times, 1250-times, 1500-times, or 2000-times greater than conventional PCR.

The currently disclosed LAMP assay for detection of T. tenax is highly specific. In some embodiments, the T. tenax LAMP assay does not detect any of the following microbes: T. vaginalis, S. pyogenes, S. aureus, E. coli, E. faecalis, and C. albicans.

The currently disclosed LAMP assay may be carried out with primers designed to detect T. tenax. A non-limiting example of primer sequences that may be used in the LAMP assay are found in TABLE 1 below. A person of skill in the art will recognize that other primers may be useful and can use methods known in the art to determine other primer sequences and optimize LAMP conditions so as to achieve the presently disclosed method. One of skill in the art will appreciate that the currently disclosed T. tenax LAMP assay may be adapted to detect other microbes, by altering the primers and optimizing conditions as disclosed herein.

The currently disclosed LAMP assay has one or more of optimized MgSO₄ concentration, temperature, and reaction time. In some embodiments, the LAMP assay has an optimized MgSO₄ concentration of about 4 mM to about 10 mM. In embodiments, the LAMP assay has an optimized MgSO₄ concentration of about 6 mM. In some embodiments, the LAMP assay has an optimized temperature of about 55° C. to about 65° C. In certain embodiments, the LAMP assay has an optimized temperature of 60° C. In some embodiments, the LAMP assay has an optimized reaction time of about 25 minutes to about 90 minutes. In certain embodiments, the LAMP assay has an optimized reaction time of 60 minutes.

The amplified products of the currently disclosed LAMP assay may be subjectively visualized using standard gel electrophoresis or by addition of SYBR Green Ito the reaction followed by examination under a UV light. A positive LAMP can also be determined by change in turbidity of the reaction solution as a result of magnesium precipitation, which may be slightly affected by an observer's objective in weakly-positive reactions.

The currently disclosed LAMP assay may be performed on any biological material. In some embodiments, the LAMP assay is performed on oral swabs. In some embodiments, the LAMP assay is performed on spiked saliva. In some embodiments, the LAMP assay is performed on urine. In some embodiments, the biological material is diluted, e.g., about 2-fold, 3-fold, 4-fold, 5-fold, 6-fold, 7-fold, 8-fold or 9-fold, or up to 10-fold or more before performing the LAMP assay.

The biological material or sample for use in any embodiments of the methods provided herein can be any nucleic acid containing material. Any nucleic acid containing material can be used as a starting material that can be directly tested for the presence or absence of T. tenax, or can be treated for extraction of polynucleotides, e.g., partly or fully isolated polynucleotides, and tested for the presence or absence of T. tenax. For example, DNA and mRNA preparations, cell extracts, tissue extracts, fluid samples (e.g., blood, serum, saliva, vaginal fluid, semen), samples from healthy and/or diseased subjects can be analyzed for the presence or absence of T. tenax according to the methods provided herein. Nucleic acid libraries also can be used as a source of starting material.

In any embodiments of the methods provided herein, the biological material or sample can be sourced from any subject to be analyzed for the presence or absence of T. tenax. The subject can be any eukaryotic species including, but not limited to, vertebrate, mammalian, human and other primates, porcine, bovine, feline, avian, equine, canine, and other eukaryotic species. In embodiments, the subject is a human. In certain embodiments, the subject is a canine.

The currently disclosed LAMP assay may be performed on a sample with or without prior DNA extraction.

Amplification products generated by any of the methods provided herein may be detected by a suitable detection process. Non-limiting examples of methods of detection include electrophoresis, nucleic acid sequencing, mass spectrometry, mass detection of mass modified amplicons (e.g., matrix-assisted laser desorption ionization (MALDI) mass spectrometry and electrospray (ES) mass spectrometry), a primer extension method (e.g., iPLEX™; Sequenom, Inc.), Molecular Inversion Probe (MIP) technology from Affymetrix, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, GOOD assay, Microarray miniseq, arrayed primer extension (APEX), Microarray primer extension, Tag arrays, coded microspheres, template-directed incorporation (TDI), fluorescence polarization, colorimetric oligonucleotide ligation assay (OLA), sequence-coded OLA, microarray ligation, ligase chain reaction, padlock probes, invader assay, High Resolution Melting (HRM), hybridization using at least one probe, hybridization using at least one fluorescently labeled probe, cloning and sequencing, the use of hybridization probes and quantitative real time polymerase chain reaction (QRT-PCR), digital PCR, nanopore sequencing, chips, MYBAIT (Arbor Biosciences), SNPCHIP, various microarray platforms, and combinations thereof.

In some embodiments, amplification products are detected using electrophoresis. Any suitable electrophoresis method, whereby amplified nucleic acids are separated by size, may be used in conjunction with the methods provided herein, which include, but are not limited to, standard electrophoretic techniques and specialized electrophoretic techniques, such as, for example capillary electrophoresis (e.g., Capillary Zone Electrophoresis (CZE), also known as free-solution CE (FSCE), Capillary Isoelectric Focusing (CIEF), Isotachophoresis (ITP), Electrokinetic Chromatography (EKC), Micellar Electrokinetic Capillary Chromatography (MECC OR MEKC), Micro Emulsion Electrokinetic Chromatography (MEEKC), Non-Aqueous Capillary Electrophoresis (NACE), and Capillary Electrochromatography (CEC)). A non-limiting standard electrophoresis example is presented as follows. After running an amplified nucleic acid sample in an agarose or polyacrylamide gel, the gel may be labeled (e.g., stained) with ethidium bromide (see, Sambrook and Russell, Molecular Cloning: A Laboratory Manual 3d ed., 2001). The presence of a band of the same size as the standard control is an indication of the presence of a target nucleic acid sequence, the amount of which may then be compared to the control based on the intensity of the band, thus detecting and quantifying the target sequence of interest. In some embodiments, where a plurality of primer pairs is used in an amplification reaction, multiple amplification products of varying size may be detected using electrophoresis.

In some embodiments of the methods provided herein, the nucleic acid can be sequenced. In some embodiments, amplified subsequences of protozoan parasites, such as T. tenax, or other microbes/pathogens (“amplification products”), can be sequenced by a sequencing process. In some embodiments, the sequencing process generates sequence reads (or sequencing reads). In some embodiments, a method herein can include identifying one or more T. tenax genotypes based on the sequence reads.

Nucleic acid may be sequenced using any suitable sequencing platform, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first-generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used. In some embodiments, a high-throughput sequencing method is used. High-throughput sequencing methods generally involve clonally amplified DNA templates or single DNA molecules that are sequenced in a massively parallel fashion, sometimes within a flow cell. Next generation (e.g., 2nd and 3rd generation) sequencing techniques capable of sequencing DNA in a massively parallel fashion can be used for methods described herein and are collectively referred to herein as “massively parallel sequencing” (MPS). In some embodiments, MPS sequencing methods utilize a targeted approach, where specific chromosomes, genes or regions of interest are sequenced. In certain embodiments, a non-targeted approach can be used, where most or all nucleic acids in a sample are sequenced, amplified and/or captured randomly.

Non-limiting examples of sequencing platforms include a sequencing platform provided by Illumina® (e.g., HiSeg™, HiSeg™ 2000, MiSeg™, Genome Analyzer™, and Genome Analyzer™ II sequencing systems); Oxford Nanopore™ Technologies (e.g., MinION sequencing system), Ion Torrent™ (e.g., Ion PGM™ and/or Ion Proton™ sequencing systems); Pacific Biosciences (e.g., PACBIO RS II sequencing system); Life Technologies™ (e.g., SOLiD sequencing system); Roche (e.g., 454 GS FLX+ and/or GS Junior sequencing systems); Helicos True Single Molecule Sequencing; Ion semiconductor-based sequencing (e.g., as developed by Life Technologies), WildFire, 5500, 5500x1 W and/or 5500x1 W Genetic Analyzer based technologies (e.g., as developed and sold by Life Technologies, U.S. Patent Application Publication No. 2013/0012399); Polony sequencing, Pyrosequencing, Massively Parallel Signature Sequencing (MPSS), RNA polymerase (RNAP) sequencing, LaserGen systems and methods, Nanopore-based platforms, chemical-sensitive field effect transistor (CHEMFET) array, electron microscopy-based sequencing (e.g., as developed by ZS Genetics, Halcyon Molecular), nanoball sequencing; or any other suitable sequencing platform. Other sequencing methods that may be used to conduct methods herein include digital PCR, sequencing by hybridization, nanopore sequencing, chromosome-specific sequencing (e.g., using DANSR (digital analysis of selected regions) technology), MYBAIT (Arbor Biosciences), SNPCHIP, and microarray platforms.

### Compositions

Provided in certain embodiments are compositions. Compositions useful for carrying out any of the methods described herein are provided. For example, compositions comprising one or more of any of the primer pairs (e.g., SEQ ID NO:6 and SEQ ID NO:7), primer sets (e.g., SEQ ID NO:2 to SEQ ID NO:7), and/or reagents for performing the methods described herein, such as PCR or LAMP, where the reagents can include, but are not limited to, probes (e.g., for qPCR, i.e., quantitative PCR), detection labels, and Mg²⁺ salts, such as MgSO₄, as described herein are provided. In certain embodiments, the compositions provided herein can include each polynucleotide or one or more polynucleotides of one or more primer pairs for detecting T. tenax by amplification, or each polynucleotide or one or more polynucleotides of one or more primer pair sets for detecting T. tenax by LAMP, which are identical, or substantially identical, to a sequence of SEQ ID NO:1, or to a subsequence of SEQ ID NO:1, or to a complement of SEQ ID NO:1, or to a complement of a subsequence of SEQ ID NO:1. In certain embodiments, each polynucleotide of one or more primer pairs for detecting T. tenax by amplification, or at least one polynucleotide of a primer pair for detecting T. tenax by amplification, or each polynucleotide of one or more primer pair sets for detecting T. tenax by LAMP, or at least one polynucleotide of a primer pair set for detecting T. tenax by LAMP, is identical, or substantially identical, to a subsequence of SEQ ID NO:1, or to a complement of a subsequence of SEQ ID NO:1, where the subsequence of SEQ ID NO:1 comprises an ITS region of T. tenax or a portion thereof, the 5.8S rRNA gene of T. tenax or a portion thereof, or any combination of regions comprising an ITS region and/or the 5.8S rRNA gene of T. tenax.

### Kits

Provided in certain embodiments are kits. The kits may include any components and compositions described herein (e.g., primers, primer pairs, primer sets (e.g., a 6-primer LAMP primer set as listed in TABLE 1), probes (e.g., for qPCR, i.e., quantitative PCR), and/or reverse complements thereof) useful for performing any of the methods described herein, in any suitable combination. Kits may further include any reagents (e.g., MgSO₄), buffers, or other components useful for carrying out any of the methods described herein. For example, a kit may include one or more primer pairs described herein and one or more components for amplifying nucleic acid, or a kit may include one or more primer sets and one or more components for performing LAMP. The kits can be configured such that a user provides a DNA template (e.g., a cDNA template) or an RNA template.

Components of a kit may be present in separate containers, or multiple components may be present in a single container. In some embodiments, primers are provided such that each container contains a single primer pair (e.g., for individual amplification reactions), or a single primer set (e.g., for individual LAMP reactions). In some embodiments, primers are provided such that one container contains a plurality of primer pairs (e.g., for multiplex amplification reactions) or a plurality of primer sets (e.g., for multiplex LAMP reactions). Suitable containers can include a single tube (e.g., vial), one or more wells of a plate (e.g., a 96-well plate, a 384-well plate, and the like), and the like.

Kits may also comprise instructions for performing one or more methods described herein and/or a description of one or more components described herein. For example, a kit may include instructions for using the primer pairs or primer sets described herein. In certain configurations, a kit may include instructions or a guide for interpreting the results of an amplification reaction, or a LAMP reaction. In certain configurations, a kit may include instructions and/or reagents for processing the biological sample prior to performing an amplification reaction, or LAMP. Instructions and/or descriptions may be in printed form and may be included in a kit insert. In some embodiments, instructions and/or descriptions are provided as an electronic storage data file present on a suitable computer readable storage medium, e.g., portable flash drive, DVD, CD-ROM, diskette, and the like. A kit also may include a written description of an internet location that provides such instructions or descriptions.

### Examples

The disclosure is described with reference to the following Examples. These Examples are provided for the purpose of illustration only and the disclosure should in no way be construed as being limited to these Examples, but rather should be construed to encompass any and all variations which become evident as a result of the disclosure provided herein.

Without further description, it is believed that one of ordinary skill in the art can, using the preceding description and the following illustrative examples, make and utilize the compounds of the present disclosure and practice the claimed methods. The following working examples are not to be construed as limiting in any way the present disclosure.

### Example 1: Development of LAMP Assay

Cells and Culture. T. tenax strain Hs-4:NIH obtained from the American Type Culture Collection (ATCC, VA, USA) was axenically grown at 35° C. for 5 days in a modified Diamonds' medium. The medium was supplemented with 10% heat-inactivated horse serum (Sigma-Aldrich, MO, USA) and penicillin and streptomycin (ThermoFisher, MA, USA). Cell density was monitored daily using a hemocytometer (Hausser Scientific, PA, USA). Genomic DNA was extracted from cultured cells using QIAGEN DNeasy Blood and Tissue Kit (QIAGEN, Hilden, Germany) according to the manufacturer's instructions and was used to optimize and assess analytical sensitivity during the development of the LAMP assay. Bacteria including Streptococcus pyogenes, S. aureus, Escherichia coli, Enterococcus faecalis and Fungi such as Candida albicans were also cultured using sheep-blood agar (BD BBL™, NJ, USA) under aerobic conditions at 37° C. for 24 hr. Trichomonas vaginalis cells originated from T. vaginalis positive urine samples diagnosed by microscopy were a gift from the Diagnostic Lab of the Joseph N. France General Hospital in St. Kitts & Nevis; no information regarding the subjects from whom the samples were obtained was provided. The T. vaginalis positive urine samples were used to assess the analytical specificity of the LAMP assay, since T. vaginalis is closely related to T. tenax.

LAMP reaction. The T. tenax genomic DNA region described in Felleisen et al., Parasitology, 115 (Pt. 2):111-119 (1997), and provided in GenBank Accession No. U86615, was targeted for the detection of T. tenax by LAMP assay. The targeted T. tenax genomic DNA sequence (SEQ ID NO:1; 368 bases), and its characterization, as described in GenBank Accession No. U86615, is set forth below:

```
(SEQ ID NO: 1)
    1 CGGTAGGTGA ACCTGCCGTT GGATCAGTTC
        TAGTTTAATA ACTAATACCA ACTTCTTTTT
  61 ATTAATCAAA AACTAATACA ATTATAAATA
        ACTAACTTCA TCAAAAATCA AGTCTCTAAG
121 CAATGGATGT CTTGGCTCCT CACACGATGA
        AGAACGTGGC ATAATGTGTT AAGTAACCGG
181 AGTTGCATAC ATCATGACAG GTTAATCTTT
        GAATGCAAAT TGCGCTAAAC TTGGCTTCGG
241 CTGAGAAGCA TGCGTGTAAC AGTACAACAT
        AATTTATAAT AATTCTTATT CTAAGCGAAT
301 AAGTAAATAA ATTATAAGAC AAACTTACGT
        TGTCTGCTAT ACGCAGGAAG ACCCGCTGAA
361 CTGAAGCA
```

The regions of the above-mentioned 368-base genomic DNA sequence of SEQ ID NO:1, as described in GenBank Accession No. U86615, are as follows:

| | |
|---|---|
| rRNA | <1..28 |
| | /product=“putative ribosomal RNA small subunit” |
| primer_bind | 1..22 |
| | /note=“TFR2” |
| misc_RNA | 29..112 |
| | /note=“putative internal transcribed spacer 1; ITS1” |
| rRNA | 113..269 |
| | /product=“putative 5.8S ribosomal RNA” |
| misc_RNA | 270..325 |
| | /note=“putative internal transcribed spacer 2; ITS2” |
| rRNA | 326..>368 |
| | /product=“putative ribosomal RNA large subunit” |
| primer_bind | complement(346..368) |
| | /note=“TFR1” |

LAMP primers targeting the ITS and 5.8S rRNA gene of T. tenax (GenBank Accession No. U86615; see regions described above) were designed using the software Primer explorer V.5 (http://primerexplorer.jp). These include FIP and BIP, F3 and B3, LF, and LB (see TABLE 1, below). Multiple sequence alignment using software CLUSTAL (Clustal Omega; https://www.ebi.ac.uk/Tools/msa/clustalo/) was carried out on the closely related protozoan T. vaginalis, to check for specificity.

TABLE 1
LAMP primer sequence for
detection of T. tenax.

| Name of Primer | Primer sequence (5′-----3′) |
|---|---|
| FIP | GTCATGATGTATGCAACTCC |
| (forward | GG-TCCTCACACGATGAAGA |
| inner | ACG (SEQ ID NO: 2) |
| primer) | |
| BIP | GGTTAATCTTTGAATGCAAA |
| (backward | TTGCG-TGTACTGTTACACG |
| inner | CATGCTTCT |
| primer) | (SEQ ID NO :3) |
| LF | ACATTATGCCACGTTCTTCA |
| (forward | TCG (SEQ ID NO:4) |
| loop | |
| primer) | |
| LB | TGCGCTAAACTTGGCTTCGG |
| (backward | (SEQ ID NO: 5) |
| loop | |
| primer) | |
| F3 | AGCAATGGATGTCTTGGC |
| (forward | (SEQ ID NO: 6) |
| outer | |
| primer) | |
| B3 | GCAGACAACGTAAGTTTGT |
| (backward | (SEQ ID NO: 7) |
| outer | |
| primer) | |

For optimization of the LAMP assay, MgSO₄ concentration, temperature and reaction time were assessed. To optimize the MgSO₄ concentration, 2 mM, 4 mM, 6 mM, 8 mM, and 10 mM MgSO₄ were used. To optimize the temperature, reactions were carried out at the following temperatures (° C.) 50, 53, 55, 58, 60, 63, or 65° C. For time optimization (minutes), 15, 30, 45, 60, and 75 minutes were used. These were all performed in Mastercycler® Nexus Gradient Cyclers (Eppendorf, Enfield, CT, USA).

The optimized LAMP reactions for the assay were performed in a 25 μl reaction mixture containing the following reagents: 0.8 μM FIP, 0.8 μM BIP, 0.4 μM LF, 0.4 μM LB, 0.20 μM F3, 0.2 μM B3, 4 mM MgSO₄, 1M betaine, 0.4 mM dNTPs, 1X thermopol buffer [20 mM Tris-HCl, 10 mM (NH₄)₂SO₄, 10 mM KCl, 2 mM MgSO₄, 0.1% Triton X-100 pH 8.8], 6 U of Bst polymerase large fragment (New England Biotechnologies, CT, USA), 2 μl of template DNA and nuclease free water. Reactions were performed at 60° C. for 60 min in the Mastercycler® Nexus Thermal Cyclers.

The amplified products were visualized using gel electrophoresis and SYBR Green I (Thermo Fisher Scientific, Waltham, MA, USA). For gel electrophoresis, 10 μl of the product was separated on 1% agarose gel containing 0.5 mg/mL ethidium bromide with a 100-bp ladder of DNA marker (Invitrogen, CA, USA). For detection using SYBR Green I, 1 μl of SYBR Green I was added to 10 μl of the product and examined under UV light. Fluorescent green color is an indication of a positive reaction and an orange color is an indication of a negative reaction.

PCR reaction. Primers F3 and B3 showed above in TABLE 1 were used in conventional PCR. The PCR was carried out in a 250 reaction mixture containing the following reagents: 2 μl template DNA, 1X Taq buffer with KCl, 0.2 mM dNTPs, 3 mM MgCl₂, 1 μM F3, 1 μM B3, 2.5 U of Taq DNA polymerase (Qiagen, MD, USA), and nuclease-free water. PCR was performed in Mastercycler® Nexus Gradient Cyclers. The thermal cycle was optimized for annealing temperature as follows: initial denaturation for 5 min at 95° C., 35 cycles of denaturation for 1 min at 95° C., annealing at 53° C. for 30 s, elongation at 72° C. for 1 min and a final step at 72° C. for 5 min.

Limit of detection and specificity. To determine the limit of detection (LD) of the LAMP assay, a 10-fold serial dilution of T. tenax genomic DNA in Tris-EDTA (TE) buffer without a carrier, ranging from 50 ng/ul to 5 pg/ul was used. The results were compared to conventional PCR using the LAMP F3 and B3 primers. Analytical specificity of the newly developed LAMP assay was evaluated using a close relative (T. vaginalis), along with other microorganisms (S. pyogenes, S. aureus, E. coli, E. faecalis, and C. albicans). Negative and positive controls using water and T. tenax genomic DNA, respectively were included. Each experiment was run three times for repeatability and reproducibility.

Analytical sensitivity of spiked samples. Cultured T. tenax cells were used to investigate the LD of the spiked samples. They were diluted in canine saliva, TE buffer, or phosphate buffered saline (PBS) in serial dilutions from 2×10⁵ to 2×10⁻¹ cells. These cells were subjected to boiling at 100° C. for 30 min followed by cooling down to 4° C. on ice. Alternatively, similar prepared spiked samples were subjected to no pre-treatment at all and were directly used to perform LAMP. Positive and negative controls using T. tenax genomic DNA and water, respectively were included. The experiment was run three times for repeatability and reproducibility.

Analysis of canine samples. LAMP was performed on eight oral swabs that were collected from client owned pet dogs that presented to the RUSVM Veterinary Clinic. These swabs were subjected to culture in modified Diamonds' medium up to 5 days as mentioned above for the presence of T. tenax. The cells were centrifuged, collected, and stored at −80° C. until use. To perform LAMP on these samples, the frozen cells were taken by a sterile pipet-tip without thawing and immediately suspended in PBS for quantification. The sample was applied directly to perform LAMP without DNA extraction or boiling after resuspension in TE buffer followed centrifugation. 2 cells per reaction were used to perform LAMP. Positive and negative controls using T. tenax genomic DNA and water, respectively were included. The experiment was run three times for repeatability and reproducibility.

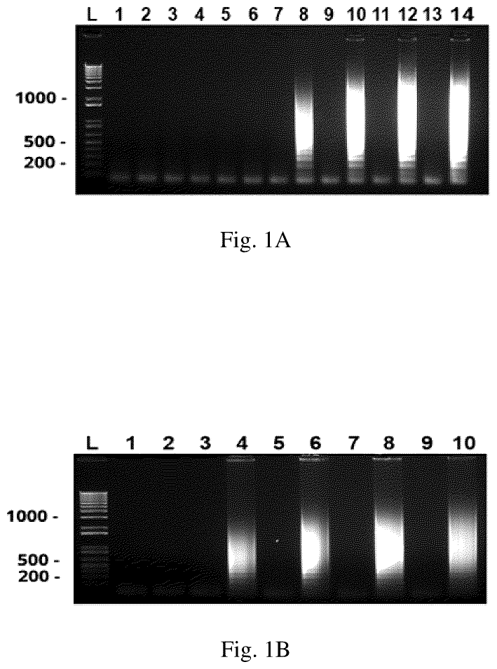

Development of LAMP assay. Reaction temperature, MgSO₄ concentration and time were optimized during the development of the LAMP assay. To optimize temperature, the reaction was incubated at the following temperatures (° C.): 50, 53, 55, 58, 60, 63, and 65° C., respectively, for 60 min. Amplification took place at the following temperatures (° C.): 58, 60, 63, and 65° C., and 60° C. was chosen as the optimal temperature based on the criteria of maximum LAMP amplification at the lowest temperature in order to extend the enzyme's half-life as long as possible. FIG. 1A depicts the temperature optimization of the LAMP reaction, with lanes 1 and 2, lanes 3 and 4, lanes 5 and 6, lanes 7 and 8, lanes 9 and 10, lanes 11 and 12, and lanes 13 and 14 are at the following temperatures (° C.): 50, 53, 55, 58, 60, 63 and 65° C., respectively. The MgSO₄ concentration was optimized by incubating reaction using (mM) 2, 4, 6, 8, and 10 mM MgSO₄, respectively. The optimal MgSO₄ concentration selected for the assay was 6 mM. To optimize the reaction time, the reaction was incubated at (minutes) 15, 30, 45, 60, and 75 minutes, respectively. Amplification was seen at 30 min and beyond, and the optimal time chosen for the assay was 60 min. FIG. 1B depicts the time optimization of the LAMP reaction, with: lanes 1 and 2, lanes 3 and 4, lanes 5 and 6, lanes 7 and 8, and lanes 9 and 10 are at 15, 30, 45, 60 and 75 min, respectively.

Therefore, all LAMP reactions from this point forward were performed at 60° C. using 6 mM MgSO₄ for 60 min.

### Example 2: Characterizing the Novel LAMP Assay

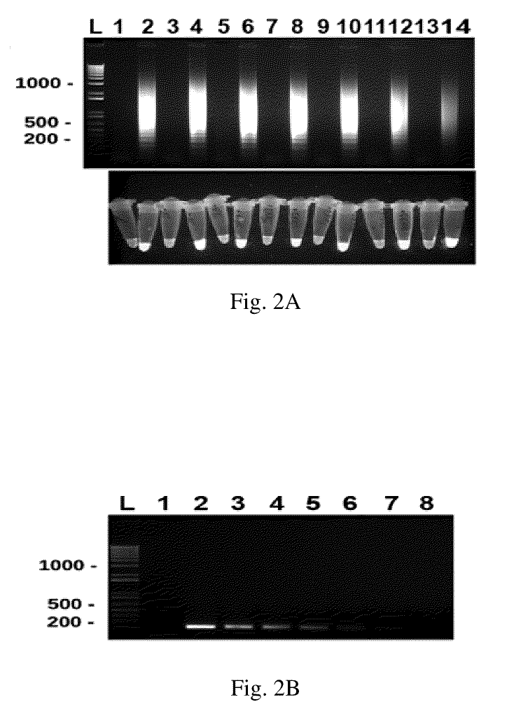

The LAMP assay described in Example 1 is more sensitive than PCR. To determine the LD of the newly developed LAMP assay, a 10-fold serial dilution of T. tenax genomic DNA with the original concentration of 50 ng/μl was used in LAMP and conventional PCR. The results of both assays were compared. LD of the LAMP assay was 10 fg T. tenax genomic DNA. This is depicted in FIG. 2A, where odd and even lanes are without and with serially diluted T. tenax genomic DNA respectively, and lanes 1 and 2, lanes 3 and 4, lanes 5 and 6, lanes 7 and 8, lanes 9 and 10, lanes 11 and 12, and lanes 13 and 14 are loaded with 10, 1, 0.1, 0.01, 0.001, 0.0001, 0.00001, and 0.000001 ng, respectively. LAMP results are detected by gel electrophoresis (top panel) and SYBR Green I with UV illumination (bottom panel of FIG. 2A). LD of conventional PCR was 10 pg T. tenax genomic DNA, which is 1,000 times higher than that of LAMP, meaning that the newly described LAMP is 1,000 times more sensitive than conventional PCR when both methods target same DNA sequence. This limit of detection using conventional PCR is depicted in FIG. 2B, where lane 1 is a negative control with water and lanes 2-8 contain 100, 10, 1, 0.1, 0.01, 0.001, and 0.0001 ng of DNA respectively. PCR results were detected by gel electrophoresis.

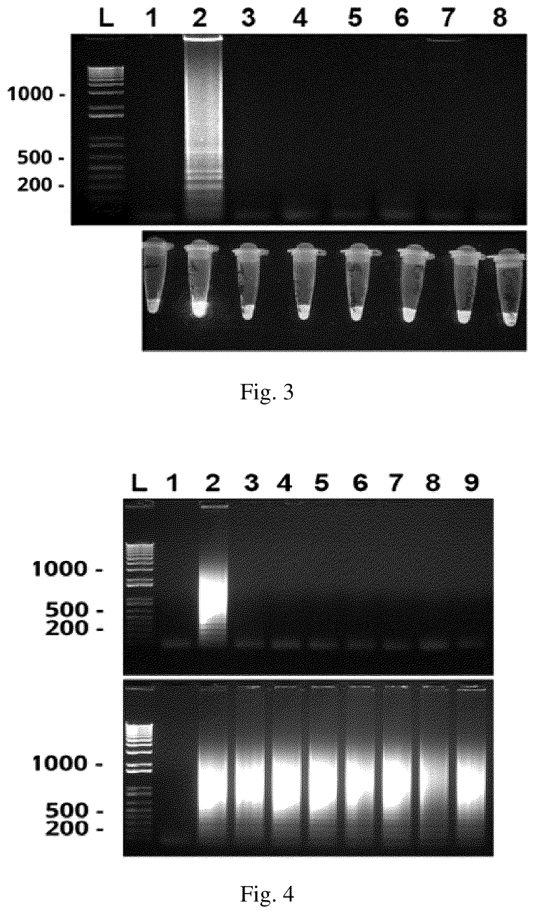

The LAMP assay described in Example 1 is more specific than PCR. To determine the specificity of the newly developed LAMP assay, genomic DNA of several microbes including T. vaginalis, S. pyogenes, S. aureus, E. coli, E. faecalis, and C. albicans were used along with that of T. tenax. As shown in FIG. 3, LAMP did not amplify any DNAs of these microbes including closely related T. vaginalis. FIG. 3 depicts in lane 1: no DNA (negative control with water); lane 2-8: containing 2 μlgenomic DNA of various microbes; lane 2: T. tenax (5ng/μl); lane 3: T. vaginalis (137 ng/μl); lane 4: S. pyogenes (74 ng/μl); lane 5: S. aureus (160 ng/μ1); lane 6: E. coli (52 ng/μl); lane 7: E. faecalis (70 ng/μl); and lane 8: C. albicans (60 ng/μl). The LAMP results are detected by gel electrophoresis (FIG. 3, top panel) and SYBR Green I with UV illumination (FIG. 3, bottom panel). These results clearly show that this LAMP assay is highly specific to T. tenax (only lane 2 containing T. tenax has amplified DNA, as seen by gel electrophoresis (FIG. 3 top panel), and only sample 2 from the left showed a bright SYBR Green I fluorescence indicating the presence of T. tenax (FIG. 3 bottom panel).

The LAMP assay described in Example 1 can detect T. tenax without prior DNA extraction. Cultured T. tenax cells were diluted in PBS or TE buffer in a 10-fold serial dilution. The diluted cells were boiled at 100° C. for 30 mins and then used directly in LAMP after being cooling down to 4° C. FIG. 4 depicts serially diluted samples of T. tenax cells in PBS (FIG. 4, top panel) or TE buffer (FIG. 4, bottom panel) boiled at 100° C. for 30 min. Lanes were loaded as follows: lanes 1 and 2: negative and positive controls without and with T. tenax genomic DNA (100 ng/μl); lanes 3-9: containing 2×10⁵, 2×10⁴, 2×10³, 2×10², 2×10¹, 2×10⁰ and 2×10⁻¹ cells respectively. As shown in FIG. 4, the newly developed LAMP assay detected T. tenax in TE buffer to as few cells as 0.2-1 cell per reaction (FIG. 4, bottom panel). In contrast, LAMP reaction was negative even with as many as 2×10⁵ T. tenax cells in PBS (FIG. 4, top panel), indicating that either PBS directly inhibits LAMP reaction or PBS does not release genomic DNA after boiling of cells.

### Example 3: Detection of T. tenax Spiked Saliva and Clinically Collected Canine Samples

Since T. tenax resides in the oral cavity of humans and canines, we next aimed to determine the limit of detection of the newly developed LAMP assay in saliva. Ethics. The collection of T. vaginalis-positive human urine was approved by RUSVM IRB (No. #20-03-XP). No information on any patients (subjects) was collected. Use of client owned dogs for the study was approved by RUSVM IACUC, with approval numbers 15-1-001 & 17.08.37 Yao. Animal care and use were in accordance with the recommendations in the Guide for the Care and Use of Laboratory Animals of the National Institute of Health.

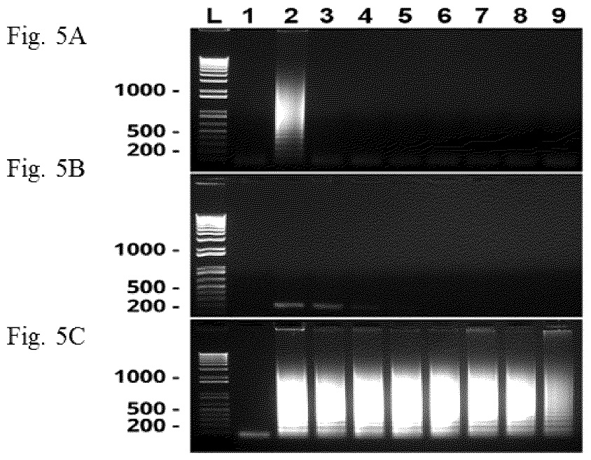

We first spiked canine saliva with cultured T. tenax using a 10-fold serial dilution ranging from 2×10⁵ to 2×10⁻1 cells. The cells were boiled at 100° C. for 30 min without DNA extraction and directly used in both LAMP and conventional PCR. The assay showed no amplification in LAMP; however, conventional PCR showed a detection limit of 2×10³ cells. This can be seen in FIG. 5A (LAMP) and FIG. 5B (PCR). For FIGS. 5A and 5B, the lanes were loaded as follows: lane 1: nuclease-free water as a negative control; lane 2: positive controls with 10Ong T. tenax genomic DNA; lanes 3-9: canine saliva spiked with 2×10⁵, 2×10⁴, 2×10³, 2×10², 2×10¹, 2×10⁰ and 2×10⁻¹ cells, respectively. They were boiled at 100° C. for 30 min with no DNA extraction. These data demonstrate that canine saliva contains some inhibitors for LAMP, but not for conventional PCR.

We next investigated whether PBS washed spiked saliva samples can be directly used without DNA extraction in LAMP. Similarly, as mentioned above, a 10-fold serial dilution of cultured T. tenax cells was spiked in saliva. The spiked samples were centrifuged, and the cell pellets were resuspended in PBS as wash. The cells were centrifuged once again and then re-suspended in TE buffer. The latter was directly used to perform LAMP. The results depicted in FIG. 5C show that the limit of detection is also 0.2 -1 cell per LAMP reaction. For FIG. 5C, the lanes were loaded as follows: lane 1: nuclease-free water as a negative control; lane 2: positive controls with 10 Ong T. tenax genomic DNA; lanes 3-9: canine saliva spiked with 2×10⁵, 2×10⁴, 2×10³, 2×10², 2×10¹, 2×10⁰ and 2'10⁻¹ cells, respectively.

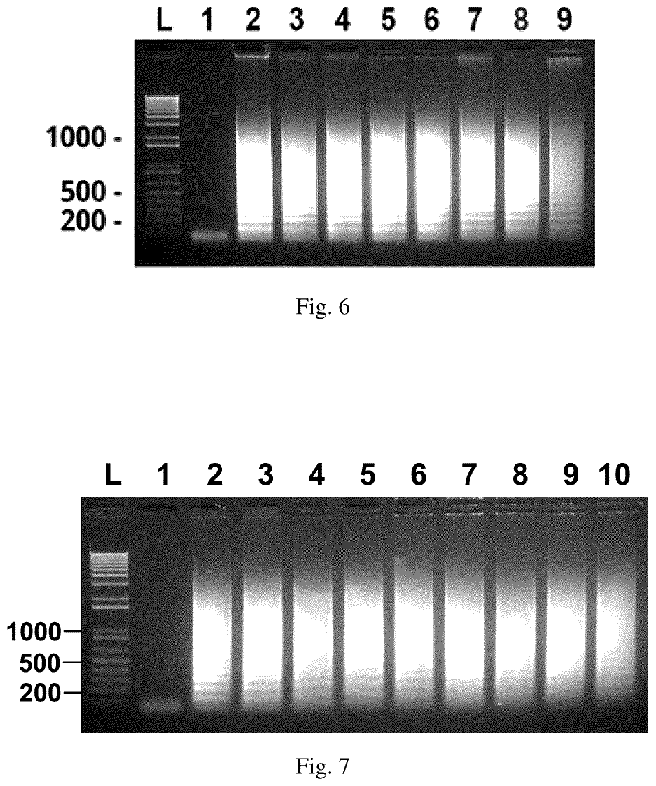

Finally, we assessed whether canine samples can be directly used in detection of T. tenax without prior DNA extraction. Oral swabs were collected along the gumline of client owned pet dogs presented to RUSVM Veterinary Clinic. From 44 samples collected, eight were positive for trichomonad protozoa by microscopy after initial culture in Diamonds' media. The cells were pelleted by centrifugation after being washed in PBS and were immediately frozen at −80° C. Cells from each of these frozen samples were picked up with a pipette-tip and re-suspended in PBS and quantified. They were then diluted in TE buffer and directly used in LAMP. FIG. 6 and FIG. 7 depict the results, with the lanes loaded as follows: lane 1: nuclease-free water as a negative control; lane 2: positive controls with 10 Ong T. tenax genomic DNA; lanes 3-10: microscopically confirmed trichomonad samples of individually owned pet dogs containing 2 cells per sample. As shown in FIG. 7, all eight microscopically confirmed trichomonad canine samples were detected positive for T. tenax. Collectively, the data demonstrates that clinical oral swabs can directly be used in the detection of T. tenax by the newly developed LAMP without prior DNA extraction. Therefore, it can be used at the point-of-care of both medical and veterinary clinics.

Moreover, the newly developed LAMP described here has been directly used to detect T. tenax in a dozen mouth samples collected from owned dogs on St. Kitts using a cotton swab. The samples were immediately preserved in 70% ethanol upon collection in the field. They were tested in a laboratory after being resuspended in TE buffer followed by a wash in PBS without DNA extraction. Several samples were detected as positive for T. tenax, which demonstrates that the LAMP presented here is capable of diagnostically detecting T. tenax at point-of care. The collected samples by cotton swabs may be stored in 70% ethanol at RT for testing performed days later or even longer. Alternatively, they may be directly put into PBS or physiological saline, followed by refrigeration for testing being performed within 24-48 hours. The expected results are used to show that the LAMP diagnostic test can be used not only at the point-of-care in both human and veterinary clinics, but also in epidemiological surveys.

### Example 4: Diagnosis of T. tenax Infections in Human Urogenital Tract

T. vaginalis is morphologically indistinguishable from T. tenax, and the two microbes cannot be distinguished with current PCR assays. However, the newly developed LAMP assay, as demonstrated above, can detect and distinguish T. tenax and T. vaginalis without ambiguity. Human urine samples submitted to the Diagnostic Lab of the Joseph N. France General Hospital in St. Kitts & Nevis, no matter whether they are microscopically positive for T. vaginalis or not, can be subjected to the T. tenax LAMP test to determine whether some clinically diagnosed T. vaginalis infections are in fact caused by T. tenax. The results can challenge the well-established view that human sexually-transmitted trichomonad disease is only caused by T. vaginalis and instead, can be caused by both T. vaginalis and T. tenax.

### Example 5: Development of T. vaginalis LAMP Assay

Primers that target the ITS and 5.8S rRNA gene of T. vaginalis have been synthesized, and studies for the detection of T. vaginalis can be performed using similar LAMP assay conditions as those described herein for T. tenax, i.e., incubation at 60° C. using 6 mM MgSO₄ for 60 min and optimization for sensitive and specific detection of T. vaginalis. This LAMP assay can be expected to be highly specific to T. vaginalis, without cross detection of T. tenax DNA.

### Example 6: Determining the Feasibility of no Freeze Conditions for LAMP Assay Reagents

Ready-to-use LAMP assay reagents can be tested for their ability to be stored under no-freeze conditions; these reagents could have the potential of not requiring storage in freezing conditions. To test this, aliquots of the LAMP reagents that include everything except samples can be placed in individual test tubes, desiccated by freeze drying (also known as lyophilization), and stored at room temperature (RT) or in refrigeration for an extended period of time. Afterward, 23 μl of nuclease free water can be used to reconstitute reagents in each tube and the latter can directly be used to test individual samples of 2 μl each. This test can help establish conditions that do not require freezing for the presently disclosed LAMP assay, thereby further facilitating ease of use of the LAMP test for end users at the point-of-care.

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

### Example 7: Examples of Embodiments

A method for detecting T. tenax comprising:
preparing a biological sample for detection;
performing LAMP on the biological sample; and
determining whether the biological sample comprises T. tenax.

The method of embodiment 1, wherein the biological sample comprises an oral swab or saliva or urine.

The method of any of embodiments 1-2, wherein the biological sample is diluted in PBS.

The method of any of embodiments 1-3, wherein neither DNA extraction nor cell boiling has been performed on the biological sample before the step of performing LAMP.

The method of any of embodiments 1-4, wherein performing LAMP comprises at least one of optimized MgSO₄ concentration, temperature, and reaction time.

The method of embodiment 5, wherein the optimized MgSO₄ concentration comprises 6 mM.

The method of embodiment 5, wherein the optimized temperature comprises 60° C.

The method of embodiment 5, wherein the optimized reaction time comprises 60 minutes.

The method of any of embodiments 1-8, wherein the determining step is at least 100-times more sensitive than conventional PCR.

The method of any of embodiments 1-9, wherein the determining step does not detect any one or more of the microbes comprising T. vaginalis, S. pyogenes, S. aureus, E. coli, E. faecalis, and C. albicans.

The method of any of embodiments 1-10, wherein the step of performing LAMP comprises use of the primers listed in TABLE 1.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described, the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

What is claimed is:

1. A method for analyzing nucleic acid from a biological sample, comprising:
contacting the biological sample, or nucleic acid obtained from the biological sample, with a set of at least two polynucleotide primer pairs under amplification conditions, thereby generating an amplified nucleic acid mixture comprising one or more amplification products; and
analyzing the amplification products, wherein one or more of the polynucleotide primer pair(s) hybridizes to subsequences of SEQ ID NO:1, if present in the nucleic acid of the biological sample, under the amplification conditions.

2. The method of claim 1, wherein analyzing the amplification products comprises determining, in the amplified nucleic acid mixture, the presence or absence of at least one amplicon that is an amplification product of a polynucleotide primer pair in the set, wherein, if the amplicon is present, then T. tenax is determined to be present in the biological sample and if the amplicon is not present, then T. tenax is determined as not being present in the biological sample.

3. The method of claim 1, wherein at least one polynucleotide primer pair comprises sequences that are identical, or substantially identical, to a subsequence of SEQ ID NO:1, or to a complement of a subsequence of SEQ ID NO:1, wherein the subsequence of SEQ ID NO:1 comprises an ITS region of T. tenax or a portion thereof, the 5.8S rRNA gene of T. tenax or a portion thereof, or any combination of regions or portions thereof comprising an ITS region and/or the 5.8S rRNA gene of T. tenax, whereby an amplified nucleic acid mixture comprising an amplification product of the subsequence of SEQ ID NO:1 is generated under the amplification conditions.

4. The method of claim 1, wherein the set of primer pairs comprises loop-mediated isothermal amplification (LAMP) primers.

5. The method of claim 4, wherein the loop-mediated isothermal amplification (LAMP) primers comprise the sequences set forth in SEQ ID NOS:2-7.

6. A method of determining the presence or absence of T. tenax in a biological sample, comprising:
contacting the biological sample, or nucleic acid obtained from the biological sample, with a set of loop-mediated isothermal amplification (LAMP) primers under amplification conditions; and
analyzing the resulting amplification products whereby, based on analyzing the amplification products, the presence or absence of T. tenax in the biological sample is determined.

7. The method of claim 6, wherein, analyzing the amplification products comprises determining the presence or absence of at least one amplicon that is an amplification product obtained by amplification of a subsequence of SEQ ID NO:1, or a complement of a subsequence of SEQ ID NO:1, wherein the subsequence of SEQ ID NO:1 comprises an ITS region of T. tenax or a portion thereof, the 5.8S rRNA gene of T. tenax or a portion thereof, or any combination of regions or portions thereof comprising an ITS region and/or the 5.8S rRNA gene of T. tenax.

8. The method of claim 7, wherein the set of loop-mediated isothermal amplification (LAMP) primers comprises the sequences set forth in SEQ ID NOS:2-7.

9. The method of claim 1, wherein the biological sample is from a human subject or the biological sample is from a canine subject.

10. The method of claim 6, wherein the biological sample is from a human subject, or the biological sample is from a canine subject.

11. The method of claim 4, wherein the amplification conditions comprise a MgSO₄ concentration of about 6 mM, a reaction temperature of about 60° C., and a reaction time of about 60 minutes.

12. The method of claim 6, wherein the amplification conditions comprise a MgSO₄ concentration of about 6 mM, a reaction temperature of about 60° C., and a reaction time of about 60 minutes.

13. The method of claim 1, wherein the biological sample comprises an oral swab, saliva or urine.

14. The method of claim 6, wherein the biological sample comprises an oral swab, saliva or urine.

15. The method of claim 6, wherein one or more of the microbes comprising T. vaginalis, S. pyogenes, S. aureus, E. coli, E. faecalis, and C. albicans, if present in the biological sample, are not detected.

16. The method of claim 6, wherein the sensitivity of determining whether T. tenax is present or absent in the biological sample is at least 100-times greater than if the biological sample, or nucleic acid obtained from the biological sample, is contacted with a primer pair for conventional PCR under amplification conditions.

17. A composition comprising a set of loop-mediated isothermal amplification (LAMP) primers comprising more than one polynucleotide primer pair, wherein each polynucleotide primer of at least one polynucleotide primer pair is selected from among a sequence that is identical, or substantially identical, to: a sequence of SEQ ID NO:1, or to a subsequence of SEQ ID NO:1, or to a complement of SEQ ID NO:1, or to a complement of a subsequence of SEQ ID NO:1.

18. The composition of claim 17, wherein at least one polynucleotide of the at least one primer pair is identical, or substantially identical, to a subsequence of SEQ ID NO:1, or to a complement of a subsequence of SEQ ID NO:1, wherein the subsequence of SEQ ID NO:1 comprises an ITS region of T. tenax or a portion thereof, the 5.8S rRNA gene of T. tenax or a portion thereof, or any combination of regions or portions thereof comprising an ITS region and/or the 5.8S rRNA gene of T. tenax.

19. The composition of claim 17, wherein the set of loop-mediated isothermal amplification (LAMP) comprises the sequences set forth in SEQ ID NOS:2-7.

20. The composition of claim 17, further comprising reagents for performing LAMP analysis.

21. The composition of claim 20, wherein the reagents for performing LAMP analysis comprises a buffer and MgSO₄.

22. A kit, comprising the composition of claim 17 and instructions for use in analyzing T. tenax nucleic acid in a biological sample.
